SEQUENCE LISTING (4 sequences)

SEQ ID NO: 1 - protein, 714 aa, homo sapiens
MSEEIITPVYCTGVSAQVQKQRARELGLGRHENAIKYLGQDYEQLRVRCLQSGTLFRDEAFPPVPQSLGYKDLGPNSSKTYGIKWKRPTELLSNPQFIVDGATRTDICQGALGDCWLLAAIASLTLNDTLLHRVVPHGQSFQNGYAGIFHFQLWQFGEWVDVVVDDLLPIKDGKLVFVHSAEGNEFWSALLEKAYAKVNGSYEALSGGSTSEGFEDFTGGVTEWYELRKAPSDLYQIILKALERGSLLGCSIDISSVLDMEAITFKKLVKGHAYSVTGAKQVNYRGQVVSLIRMRNPWGEVEWTGAWSDSSSEWNNVDPYERDQLRVKMEDGEFWMSFRDFMREFTRLEICNLTPDALKSRTIRKWNTTLYEGTWRRGSTAGGCRNYPATFWVNPQFKIRLDETDDPDDYGDRESGCSFVLALMQKHRRRERRFGRDMETIGFAVYEVPPELVGQPAVHLKRDFFLANASRARSEQFINLREVSTRFRLPPGEYVVVPSTFEPNKEGDFVLRFFSEKSAGTVELDDQIQANLPDEQVLSEEEIDENFKALFRQLAGEDMEISVKELRTILNRIISKHKDLRTKGFSLESCRSMVNLMDRDGNGKLGLVEFNILWNRIRNYLSIFRKFDLDKSGSMSAYEMRMAIESAGFKLNKKLYELIITRYSEPDLAVDFDNFVCCLVRLETMFRFFKTLDTDLDGVVTFDLFKWLQLTMFA

SEQ ID NO: 2 - DNA, 3007 bp, homo sapiens
aaggagagagggagggcggagggcggaggggcggcgggaggagggcggggaggagcgctcttcctggttgggccctgccctgagctgccaccgggaagccagcctcagggactgcagcgacccccaaacacccctcccccaggatgtcggaggagatcatcacgccggtgtactgcactggggtgtcagcccaagtgcagaagcagcgggccagggagctgggcctgggccgccatgagaatgccatcaagtacctgggccaggattatgagcagctgcgggtgcgatgcctgcagagtgggaccctcttccgtgatgaggccttccccccggtaccccagagcctgggttacaaggacctgggtcccaattcctccaagacctatggcatcaagtggaagcgtcccacggaactgctgtcaaacccccagttcattgtggatggagctacccgcacagacatctgccagggagcactgggggactgctggctcttggcggccattgcctccctcactctcaacgacaccctcctgcaccgagtggttccgcacggccagagcttccagaatggctatgccggcatcttccatttccagctgtggcaatttggggagtgggtggacgtggtcgtggatgacctgctgcccatcaaggacgggaagctagtgttcgtgcactctgccgaaggcaacgagttctggagcgccctgcttgagaaggcctatgccaaggtaaatggcagctacgaggccctgtcagggggcagcacctcagagggctttgaggacttcacaggcggggttaccgagtggtacgagttgcgcaaggctcccagtgacctctaccagatcatcctcaaggcgctggagcggggctccctgctgggctgctccatagacatctccagcgttctagacatggaggccatcactttcaagaagttggtgaagggccatgcctactctgtgaccggggccaagcaggtgaactaccgaggccaggtggtgagcctgatccggatgcggaacccctggggcgaggtggagtggacgggagcctggagcgacagctcctcagagtggaacaacgtggacccatatgaacgggaccagctccgggtcaagatggaggacggggagttctggatgtcattccgagacttcatgcgggagttcacccgcctggagatctgcaacctcacacccgacgccctcaagagccggaccatccgcaaatggaacaccacactctacgaaggcacctggcggcgggggagcaccgcggggggctgccgaaactacccagccaccttctgggtgaaccctcagttcaagatccggctggatgagacggatgacccggacgactacggggaccgcgagtcaggctgcagcttcgtgctcgcccttatgcagaagcaccgtcgccgcgagcgccgcttcggccgcgacatggagactattggcttcgcggtctacgaggtccctccggagctggtgggccagccggccgtacacttgaagcgtgacttcttcctggccaatgcgtctcgggcgcgctcagagcagttcatcaacctgcgagaggtcagcacccgcttccgcctgccacccggggagtatgtggtggtgccctccaccttcgagcccaacaaggagggcgacttcgtgctgcgcttcttctcagagaagagtgctgggactgtggagctggatgaccagatccaggccaatctccccgatgagcaagtgctctcagaagaggagattgacgagaacttcaaggccctcttcaggcagctggcaggggaggacatggagatcagcgtgaaggagttgcggacaatcctcaataggatcatcagcaaacacaaagacctgcggaccaagggcttcagcctagagtcgtgccgcagcatggtgaacctcatggatcgtgatggcaatgggaagctgggcctggtggagttcaacatcctgtggaaccgcatccggaattacctgtccatcttccggaagtttgacctggacaagtcgggcagcatgagtgcctacgagatgcggatggccattgagtcggcaggcttcaagctcaacaagaagctgtacgagctcatcatcacccgctactcggagcccgacctggcggtcgactttgacaatttcgtttgctgcctggtgcggctagagaccatgttccgatttttcaaaactctggacacagatctggatggagttgtgacctttgacttgtttaagtggttgcagctgaccatgtttgcatgaggcagggactcggtcccccttgccgtgctcccctccctcctcgtctgccaagcctcgcctcctaccacaccacaccaggccaccccagctgcaagtgccttccttggagcagagaggcagcctcgtcctcctgtcccctctcctcccagccaccatcgttcatctgctccgggcagaactgtgtggcccctgcctgtgccagccatgggctcgggatggactccctgggccccacccattgccaagccaggaaggcagctttcgcttgttcctgcctcgggacagccccgggtttccccagcatcctgatgtgtcccctctccccacttcagaggccacccactcagcaccaccggcctggccttgcctgcagactataaactataaccactagctcgacacagtctgcagtccaggcgtgtggagccgcctcccggctcggggaggccccggggctgggaacgcctgtgccttcctgcgccgaagccaacgccccctctgtccttccctggccctgctgccgaccaggagctgcccagcctgtgggcggtcggccttccctccttcgctccttttttatattagtgattttaaaggggactcttcagggacttgtgtactggttatgggggtgccagaggcactaggcttggggtggggaggtcccgtgttccatatagaggaaccccaaataataaaaggccccacatctgtctgtg

SEQ ID NO: 3 - protein, 700 aa, homo sapiens
MAGIAAKLAKDREAAEGLGSHERAIKYLNQDYEALRNECLEAGTLFQDPSFPAIPSALGFKELGPYSSKTRGMRWKRPTEICADPQFIIGGATRTDICQGALGDCWLLAAIASLTLNEEILARVVPLNQSFQENYAGIFHFQFWQYGEWVEVVVDDRLPTKDGELLFVHSAEGSEFWSALLEKAYAKINGCYEALSGGATTEGFEDFTGGIAEWYELKKPPPNLFKIIQKALQKGSLLGCSIDITSAADSEAITFQKLVKGHAYSVTGAEEVESNGSLQKLIRIRNPWGEVEWTGRWNDNCPSWNTIDPEERERLTRRHEDGEFWMSFSDFLRHYSRLEICNLTPDTLTSDTYKKWKLTKMDGNWRRGSTAGGCRNYPNTFWMNPQYLIKLEEEDEDEEDGESGCTFLVGLIQKHRRRQRKMGEDMHTIGFGIYEVPEELSGQTNIHLSKNFFLTNRARERSDTFINLREVLNRFKLPPGEYILVPSTFEPNKDGDFCIRVFSEKKADYQAVDDEIEANLEEFDISEDDIDDGVRRLFAQLAGEDAEISAFELQTILRRVLAKRQDIKSDGFSIETCKIMVDMLDSDGSGKLGLKEFYILWTKIQKYQKIYREIDVDRSGTMNSYEMRKALEEAGFKMPCQLHQVIVARFADDQLIIDFDNFVRCLVRLETLFKIFKQLDPENTGTIELDLISWLCFSVL

SEQ ID NO: 4 - DNA, 3419 bp, homo sapiens
ttccctccggtgaatcatcgctcgcagcggcggcgcccgcagtggccgcagcagcgcgccgggccctggccgcgccccagccgagcgcagcgcggagtcgccccgacctttctctgcgcagtacggccgccgggaccgcagcatggcgggcatcgcggccaagctggcgaaggaccgggaggcggccgaggggctgggctcccacgagagggccatcaagtacctcaaccaggactacgaggcgctgcggaacgagtgcctggaggccgggacgctcttccaggacccgtccttcccggccatcccctcggccctgggcttcaaggagttggggccctactccagcaaaacccggggcatgagatggaagcgccccacggagatctgcgctgacccccagtttatcattggaggagccacccgcacagacatctgccaaggagccctaggtgactgctggctgctggcagccattgcctccctcaccttgaatgaagaaatcctggctcgagtcgtccccctaaaccagagcttccaggaaaactatgcagggatctttcacttccagttctggcaatacggcgagtgggtggaggtggtggtggatgacaggctgcccaccaaggacggggagctgctctttgtgcattcagccgaagggagcgagttctggagcgccctgctggagaaggcatacgccaagatcaacggatgctatgaagctctatcagggggtgccaccactgagggcttcgaagacttcaccggaggcattgctgagtggtatgagttgaagaagccccctcccaacctgttcaagatcatccagaaagctctgcaaaaaggctctctccttggctgctccatcgacatcaccagcgccgcggactcggaggccatcacgtttcagaagctggtgaaggggcacgcgtactcggtcaccggagccgaggaggttgaaagtaacggaagcctacagaaactgatccgcatccgaaatccctggggagaagtggagtggacagggcggtggaatgacaactgcccaagctggaacactatagacccagaggagagggaaaggctgaccagacggcatgaagatggagaattctggatgtctttcagtgacttcctgaggcactattcccgcctggagatctgtaacctgaccccagacactctcaccagcgatacctacaagaagtggaaactcaccaaaatggatgggaactggaggcggggctccaccgcgggaggttgcaggaactacccgaacacattctggatgaaccctcagtacctgatcaagctggaggaggaggatgaggacgaggaggatggggagagcggctgcaccttcctggtggggctcattcagaagcaccgacggcggcagaggaagatgggcgaggacatgcacaccatcggctttggcatctatgaggttccagaggagttaagtgggcagaccaacatccacctcagcaaaaacttcttcctgacgaatcgcgccagggagcgctcagacaccttcatcaacctccgggaggtgctcaaccgcttcaagctgccgccaggagagtacattctcgtgccttccaccttcgaacccaacaaggatggggatttctgcatccgggtcttttctgaaaagaaagctgactaccaagctgtcgatgatgaaatcgaggccaatcttgaagagttcgacatcagcgaggatgacattgatgatggagtcaggagactgtttgcccagttggcaggagaggatgcggagatctctgcctttgagctgcagaccatcctgagaagggttctagcaaagcgccaagatatcaagtcagatggcttcagcatcgagacatgcaaaattatggttgacatgctagattcggacgggagtggcaagctggggctgaaggagttctacattctctggacgaagattcaaaaataccaaaaaatttaccgagaaatcgacgttgacaggtctggtaccatgaattcctatgaaatgcggaaggcattagaagaagcaggtttcaagatgccctgtcaactccaccaagtcatcgttgctcggtttgcagatgaccagctcatcatcgattttgataattttgttcggtgtttggttcggctggaaacgctattcaagatatttaagcagctggatcccgagaatactggaacaatagagctcgaccttatctcttggctctgtttctcagtactttgaagttataactaatctgcctgaagacttctcatgatggaaaatcagccaaggactaagcttccatagaaatacactttgtatctggacctcaaaattatgggaacatttacttaaacggatgatcatagctgaaaataatgatactgtcaatttgagatagcagaagtttcacacatcaaagtaaaagatttgcatatcattatactaaatgcaaatgagtcgcttaacccttgacaaggtcaaagaaagctttaaatctgtaaatagtatacactttttacttttacacactttcctgttcatagcaatattaaatcaggaaaaaaaaatgcagggaggtatttaacagctgagcaaaaacattgagtcgctctcaaaggacacgaggcccttggcagggaatatttaaagcaacttcaagtttaaaatgcagctgttgattctaccaaacaacagtccaagattaccatttcccatgagccaactgggaaacatggtatatcatgaagtaatcttgtcaaggcatctggagagtccaggagaggagactcacctctgtcgcttgggttaaacaagagacaggttttgtagaatattgattggtaatagtaaatcgttctccttacaatcaagttcttgaccctattcggccttatacatctggtcttacaaagaccaaagggatcctgcgcttgatcaactgaaccagtatgccaaaaccaggcatccaatttgtaaaccaattatgataaaggacaaaataagctgtttgccacctcaaaactttatgaacttcaccaccactagtgtctgtccatggagttagaggggacatcacttagaagttcttatagaaaggacacaagtttgtttcctggctttaccttgggaaaatgctagcaacattatagaaattttgccttgttgccttatcttcttccaaatgtactgttaaataaaaataaagggttaccccatgcaatcacaccatgccatgttttccttcctggagggcagccccacaggacggtttatgagcacacaattatagcttgtttctactttaacagggtatgctgcctctgtaaattcatgtattcaaaggaaaagacaccttgcctataattaaaatgtggaactataaaattttttaaaatccaaaaaaaaaaaaaaa

(19) United States
(12) Patent Application Publication
(10) Pub. No.: US 2005/0014182 A1
(43) Pub. Date: Jan. 20, 2005

(54) Use of calpain for identifying compounds that modulate pain

(75) Inventors: Martin Michaelis, Frankfurt (DE); Gerd Geisslinger, Bad Soden (DE); Ellen Niederberger, Frankfurt (DE)

Correspondence Address: F. Aaron Dubberley;Aventis Pharmaceuticals Inc., Patent Department, Route #202-206 / P.O. Box 6800, Bridgewater, NJ, 08807-0800, US

(73) Assignee: Aventis Pharma Deutschland GmbH, Frankfurt am Main (DE)

(21) Appl. No.: 10/886,451

(22) Filed: Jul. 7, 2004

Related U.S. Application Data
(60) Provisional application No. 60/528,375, filed on Dec. 10, 2003.

(30) Foreign Application Priority Data
Jul. 14, 2003 (DE) 10331980.8-41

(51) Int. Cl. C12Q 1/68; C12Q 1/37

(52) U.S. Cl. 435/6; 435/23

(57) ABSTRACT

The invention relates to the use of calpain or functional fragments or derivatives thereof for the preparation of pharmaceutical compounds that modulate pain, and the use of calpain or functional fragments of derivatives thereof for identifying such compounds, and a method of screening pharmaceuticals useful for modulating and/or preventing pain.

### FIELD OF THE INVENTION

The invention relates to the use of calpain or functional fragments or derivatives thereof for the preparation of pharmaceutical compounds that modulate pain, and the use of calpain or functional fragments of derivatives thereof for identifying such compounds, and a method of screening pharmaceuticals useful for modulating and/or preventing pain.

### DESCRIPTION OF THE INVENTION

The effects of peripheral inflammatory stimulation on the protein expression pattern in the rat spinal cord were investigated. By the method of two-dimensional (2D) gel electrophoresis a time-dependent breakdown of neurofilament light chain (NF-L) protein after 24, 48 and 96 h of zymosan treatment was determined, which was corroborated by immunohistochemistry and Western Blot experiments.

Since neurofilaments are substrates of the calcium-dependent cysteine protease calpain the role of calpain in inflammatory and painful processes was further assessed. It could be shown that peripheral inflammatory stimulation with zymosan leads to an upregulation of calpain activity and protein expression in the rat spinal cord. Inhibition of calpain proteolysis using the specific calpain inhibitor III (MDL-28170; 12.5 mg/kg and 25 mg/kg i.p.) attenuated the zymosan induced neurofilament degradation. Calpain protein expression was not affected by MDL-28170. The chemical structure of calpain inhibitor III is as follows:

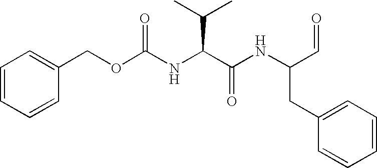

The effects of MDL-28170 on inflammation and nociception have been tested in two different animal models. In the zymosan induced paw inflammation model calpain inhibitor III (25 mg/kg) significantly reduced the paw edema compared to control animals. In the thermal hyperalgesia model application of calpain inhibitor III led to antinociceptive responses. Taken together, these data suggest an important role of calpain in inflammatory and painful processes and therefore indicate a therapeutic potential of calpain inhibitors as alternative antiinflammatory and analgesic drugs.

Additionally, calpain inhibitor MDL-102935 has been tested as described for calpain inhibitor III. The chemical formula of MDL-102935 is the following:

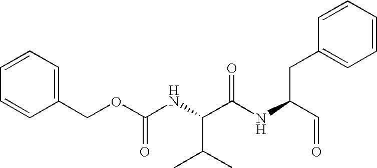

The effects of MDL-102935 on inflammation and nociception are the same as those of MDL-28170. MDL-102935 is an inhibitor of calpain.

In spinal nociceptive processing various painful stimuli from the periphery are switched over from the primary afferents to the central nervous system in the dorsal horn of the lumbal spinal cord. Persistent stimulation of primary nociceptors and C-fibres, as occurring with inflammatory stimuli triggers an increased excitability of nociceptive neurons in the spinal cord and thereby results in hyperalgesia and allodynia (Yaksh et al., 1999). Long term alterations in synaptic communication (so called long term potentiation (LTP)) are associated with processes of learning and memory (Bliss et al., 1993) and may be responsible for the synaptic plasticity and chronic pain (Rygh et al., 1999; Vikman et al., 2001). Repeated or prolonged noxious stimulation activate a number of intracellular second messenger systems, implying phosphorylation of key membrane receptors and channels by protein kinases, particularly protein kinase C (PKC). This kinase activation results in posttranslational changes that can lead to an increase in synaptic efficacy. Intracellular signal cascades result in an induction of immediate early genes (IEG) (c-fos, Zif 268, Cox-2), and later response genes encoding neuropeptides, neuropeptide and neurotrophin receptors. This protein alterations are considered as the beginning of a widespread change in protein synthesis and furthermore as a general basis for nervous system plasticity (Dubner et al., 1992; Ji et al., 1994; McCarson et al., 1994) (Beiche et al., 1996; Ji et al., 1995; Mannion et al., 1999) (Woolf et al., 1999). In the current study intracellular changes of the protein expression pattern accompanying peripheral inflammatory conditions within the rat lumbal spinal cord have been investigated by two-dimensional gel electrophoresis (2D PAGE) combined with MALDI-TOF MS. Using this method we detected several protein alterations in the rat zymosan induced paw inflammation model. In this study it was focussed on one of them which was identified as neurofilament light chain (NF-L). NF-L is a cytoskeleton protein occuring in neuronal cells where it is responsible for correct assembly of neurofilaments and maintenance of axonal calibre (Sakaguchi et al., 1993). Neurofilament desorganisation is associated with a variety of neurodegenerative diseases like Parkinson and amyotrophic lateral sclerosis (Julien, 1999). Moreover neuronal death and neurological dysfunction after spinal cord injury comes along with the loss of neurofilament protein (Ray et al., 2000a). The degradation of neurofilament proteins is mostly mediated by the action of the protease calpain (Ray et al., 2000b; Schlaepfer et al., 1985; Stys et al., 2002). Calpains are a family of ubiquitously expressed calcium dependent cysteine proteases that have been implicated in basic cellular processes including proliferation, apoptosis and differentiation. Many calpain substrates are localized in the pre- and postsynaptic compartments of neurons indicating growing evidence for the involvement of calpains in neurodegenerative diseases and in the modulation of synaptic plasticity (Chan et al., 1999). The calpain of rat is described by Thompson and Goll (Meth. Mol. Biol., Vol 144, 3-16, 2000). Two human calpain isoforms are known, namely calpain I (embl locus HSCANPR, accession no. X04366.1) and calpain II (Morford et al., Biochem. Biophys. Res. Commun. 295 (2), 540-546, 2002).

In the current study the role of calpain in the zymosan induced neurofilament degradation as well the potential anti-inflammatory and antinociceptive properties of a specific calpain inhibitor (MDL 28170) in the well established zymosan-induced paw inflammation model was investigated.

Surprisingly it has now been found that calpain plays a role in connection with pain.

An embodiment of the invention is the use of calpain or functional fragments or derivatives thereof for the preparation of pharmaceutical compounds that modulate pain.

A functional fragment of derivative of calpain is one that retains the protease activity of calpain.

Another embodiment of the invention is the use of calpain or functional fragments of derivatives thereof for identifying compounds that modulate pain.

A further embodiment of the invention is the use as described above, wherein the pharmaceutical compounds are compounds for the prevention or treatment of pain, in particular, wherein the compounds lessen or abolish pain.

Another embodiment of the invention is a method of screening pharmaceuticals useful for modulating and/or preventing pain, comprising the steps
a. Providing two samples
b. contacting one sample containing calpain or a functional fragment or derivative thereof with a compound,
c. determining the calpain activity in the presence of compound,
d. determining the calpain activity in the absence of compound, and
e. comparing the calpain activity according to c) with that according to d).

Another embodiment of the invention is the use or method as described above, wherein calpain is human calpain I and/or calpain II, in particular wherein calpain is an isolated polypeptide or polynucleotide.

Another embodiment of the invention is a use or a method as described above, wherein calpain is a polypeptide, in particular wherein calpain is a polypeptide or functional fragment thereof that comprises or consists of the sequence according to SEQ ID No 1 (calpain 1) or a fragment thereof, the sequence according to SEQ ID No 3 (calpain II) or a fragment thereof, or is encoded by a polynucleotide comprising or consisting of the sequence according to SEQ ID No 2 (encoding calpain II) or a fragment thereof, or of the sequence according to SEQ ID No 4 (encoding calpain II) or a fragment thereof.

Another embodiment of the invention is a use or a method as described above, wherein calpain is a polynucleotide, in particular wherein calpain is a polynucleotide comprising or consisting of the sequence or a part of the sequence coding for a functional fragment of calpain according to SEQ ID No 2 or SEQ ID No 4 or a polynucleotide comprising or consisting of a sequence that is able to hybridize with said polynucleotides under stringent conditions, preferably wherein the functional fragments comprise or consist of amino acids 115 to 272 of SEQ ID No 2, and/or 105 to 262 of SEQ ID No 4.

Another embodiment of the invention is a method as described above, wherein a cell expressing calpain, preferably expressing recombinant calpain, is used.

Another embodiment of the invention is a method as described above, wherein a modified cell, having a lower calpain activity as compared to its unmodified state, is used, preferably wherein a calpain knock-out cell is used.

Another embodiment of the invention is a method as described above, wherein the calpain activity is determined directly or indirectly.

A further embodiment of the invention is a method of identifying a compound that modulates pain comprising
a. Selecting a compound that modulates the activity of calpain as a test compound, and
b. Administering said test compound to a subject to determine whether the pain is modulated.

A further embodiment of the invention is the use of MDL-28170 (calpain inhibitor III) in the process of preparing a medicament for the treatment of pain.

A further embodiment of the invention is the use of MDL-28170 as a medicament for the treatment of pain.

A further embodiment of the invention is the use of MDL-102935 in the process of preparing a medicament for the treatment of pain.

A further embodiment of the invention is the use of MDL-102935 as a medicament for the treatment of pain.

In the following the invention is described by the examples which shall in no way limit the invention to the specific embodiments of the examples.

### BRIEF DESCRIPTION OF THE FIGURES

FIG. 1: Zymosan treatment causes a time-dependent decrease of Neurofilament-L
(A) Enlarged regions of 2D gels of control rat spinal cord tissue and after 24 h, 48 h, and 96 h zymosan treatment. Arrows indicate protein 1, which was identified as neurofilament light chain.
(B) Representative western blot analysis of neurofilament light chain (NF-L) expression following zymosan treatment for 24, 48, and 96 h.
(C) Densitometric analysis of 3 independent Western Blot experiments
(**statistically significant mean difference, p<0.01).

FIG. 2: Calpain activity is elevated after zymosan treatment and reduced by calpain inhibitor III
(A): Calpain activity of control rat spinal cord extracts and after 24, 48 and 96 h zymosan treatment.
(B): Calpain activity of spinal cord extracts of zymosan treated control rats and animals that received either 12.5 mg/kg or 25 mg/kg calpain inhibitor III.
The activity is determined by the fluometric detection of the cleavage of the calpain substrate Ac-LLY-AFC.
(*, **statistically significant mean difference p<0.01; p<0.05, respectively)

FIG. 3: Immunohistochemical Cy-3 labeling of lumbal spinal cord sections. Cross sections were taken of control animals as well as animals 96 h after injection of zymosan with and without pre-treatment with calpain inhibitor II. The images represent the dorsal horn of the spinal cord.
(A): Sections stained immunohistochemically with monoclonal anti-NF-L antibody. Calpain inhibitor III reduced the loss of NF-L after zymosan treatement.
(B): Sections stained immunohistochemically with monoclonal anti-m-calpain antibody. Zymosan treatment led to an increased m-calpain immunopositivity, which was not altered by calpain inhibitor III.

FIG. 4: Calpain inhibitor III significantly reduces the paw oedema at 25 mg/kg
(A): Time course of the alteration of the paw volume after intraplantar injection of 1.25 mg zymosan in control animals (♦) and in rats treated with 12.5 (▪), and 25 mg/kg calpain inhibitor III (▴).
(B): For statistical comparison of drug effects the areas under the “paw volume increase” versus “time” curves (AUC_{ΔPW} from 0-8 h, mean±s.e.m.) were calculated using the linear trapezoidal rule and subjected to univariate analysis of variance with subsequent Bonferroni post hoc tests. (* statistically significant mean difference with p<0.05)

FIG. 5: Calpain inhibitor III significantly reduces thermal hyperalgesia at 25 mg/kg. Time course of paw withdrawal latency in response to thermal stimulation of the plantar surface after intraplantar injection of 1.25 mg zymosan alone (♦) or zymosan and calpain inhibitor III (▴). Data are expressed as the relative difference between the zymosan treated right and the untreated left hind paw calculated as: ΔPWL=(right−left)/left×100.
(A): Paw withdrawal latency after intraperitoneal administration of 25 mg/kg calpain inhibitor III in comparison with vehicle treated rats.
(B): Area under the effect (relative decrease of paw withdrawal latency, ΔPWL) versus time curves after intraplantar injection of zymosan and treatment with 25 mg/kg Calpain inhibitor III. (***, statistically significant mean difference, p<0.001)

FIG. 6: Scheme of the potential signal transduction pathway in the dorsal horn of the spinal cord

### EXAMPLES

Materials and Methods Used in the Examples:

Animals

Male Sprague Dawley rats (Charles River, Sulzbach, Germany) weighing 260-300 g were housed in groups of five in standard cages and maintained in climate- and light-controlled rooms (22±0.5° C., 12/12 h dark/Light cycle) with free access to food and water. In all experiments the ethic guidelines for investigations in conscious animals were obeyed and the procedures approved by the local Ethics Committee.

Zymosan-Treatment of Rats

Unilateral hind paw inflammation and paw oedema was induced by subcutaneous injection of 1.25 mg zymosan (Sigma, München, Germany), suspended in 100 μl phosphate buffered saline (12.5 mg/ml), into the midplantar region of the right hind paw. After 24, 48 or 96 h animals were anaesthetized and killed by cardiac puncture. Spinal cords were excised, directly frozen in liquid nitrogen and then kept at −80° C. until further analysis.

Paw Oedema

Paw oedema was induced by zymosan injection as described above. The paw volume was measured before and 0.25, 0.5, 1, 2, 4, 6, 8 and 24 h after zymosan injection using a plethysmometer (Ugo Basile, Varese, Italy). Six rats per group were used. At each time point five measurements of the paw volume were taken. The average of these five measurements was used for further analysis.

Thermal Hyperalgesia

Nociceptive paw withdrawal latency to radial heat was assessed according to Hargreaves (Hargreaves et al., 1988), using a commercially available special device (Ugo Basile, Varese, Italy). Rats were placed in in transparent plastic chambers (18×29×12.5 cm) on a metal grid (5×5 mm). The heat source consisting of a high projector lamp bulb was focused on the mid-plantar surface of the hind paw. The bulb and an electronic timer were simultaneously activated at the test start, and both were automatically inactivated when a photocell detected a paw withdrawal in response to the heat. Paw withdrawal latencies (PWL) of the right and left paw (treated and untreated paw) were recorded before and every 60 min up to 8 h and 24 h after zymosan injection.

Drug Treatment

Calpain inhibitor III (MDL 28170) (Calbiochem, Schwalbach/Ts., Germany) was dissolved in PEG 400/DMSO (1:1) at a concentration of 10 mg/ml. Animals received calpain inhibitor III either as a 12.5 mg/kg or a 25 mg/kg intraperitoneal (i.p.) bolus injection 20 min prior to the intraplantar injection of zymosan. Six animals were used for each treatment group. (Controls received 1 ml of vehicle.) Treatments were randomly allocated to animals and the observers were unaware of treatment allocations.

Data Analysis and Statistics

The percent relative difference in paw withdrawal latency (ΔPWL) between the zymosan treated right and the untreated left hind paw, calculated as: ΔPWL=(right−left)/left×100, was used to asses antinociceptive effects. To evaluate anti-inflammatory effects, the relative difference between the paw volume before and after zymosan injection (ΔPV) was used. The areas under the ΔPWL versus time curves (AUC_{ΔPWL}) as well as the areas under the ΔPV versus time curves (AUC_{ΔPV}) were calculated using the linear trapezoidal rule. Statistical evaluation was done by SPSS 9.02 for Windows. Effects of study medications were assessed by submitting the AUC_{ΔPV} from 0-8 h to univariate analysis of variance (ANOVA) with subsequent t-tests employing a Bonferroni □-correction for multiple comparisons. Statistical significance of ΔPWL was calculated by unpaired Student's t-test (zymosan-treated rats versus control rats). □ was set at 0.05.

2D PAGE

Lumbal spinal cords were homogenized in lysis buffer containing 9 M urea, 2% CHAPS, 1% DTT and 2 mM Pefabloc. After removal of cellular debris extracts were ultracentrifuged at 40'000 rpm for 1 h (15° C.) and the supernatent was stored at −70° C. till further analysis. Protein concentrations were determined by the Bradford protein assay.

2D PAGE was performed as described previously (Gorg et al., 1995) with slight modifications. In brief, for the first dimension (isoelectric focusing (IEF)) 600 μg of protein were precipitated with 50% TCA at −20° C. The pellet was redissolved in rehydration solution (8 M urea, 2% CHAPS, 2.8% DTT and 0.5% IPG buffer (Amersham Biosciences, Freiburg, Germany)) and applied to 13 cm Immobiline DryStrips, pH 3-10 linear (Amersham Biosciences, Freiburg, Germany). IEF was performed using an Amersham IPGphor isoelectric focusing system and the run was carried out as follows: Rehydration 2 h, 30 V for 6 h, 60 V for 3 h, 200 V for 1 h, 500 V for 1 h, 5000 V for 4 h and 8000 V for 1 h. After IEF the IPG strips were equilibrated for 10 min in equilibration buffer (1.5 M Tris-Cl pH 8.8, 6 M urea, 30% glycerol, 2% SDS) with addition of 10% DTT and then for further 10 min in equilibration buffer containing 260 mM iodacetamide.

The IPG strips were then placed on the top of a 13% sodium dodecyl sulfate-polyacrylamid gel and sealed with 1% agarose solution. Electrophoresis in the second dimension was performed at 2.5 W/gel for 1 h followed by 5 W/gel for 4 h. Gels were stained with Coomassie Brilliant Blue. The stained gels were imaged on a UMAX PowerLookill scanner and analysed with Image Master 2D software (Amersham Biosciences, Freiburg, Germany). Coomassie stained spots of interest were cut out of the gels, proteins were digested and then identified by peptide mass fingerprinting using the matrix-assisted laser desorption ionisation time of flight mass spectrometry (MALDI-TOF MS).

Immunoblot Analysis

Equivalent amounts of total cellular proteins, extracted from homogenized spinal cords, were diluted with 4× sample buffer (1× sample buffer consists of 2.5% sodium dodecyl sulfate (SDS), 50 mM Tris-HCl (pH 6.8), 2.5% 2-mercaptoethanol, 10% glycerol and a trace of Bromphenol Blue). Samples were boiled for 5 min, and proteins (30 μg per lane) were subjected to SDS-PAGE using 10% polyacrylamide gels. Proteins were then transferred onto nitrocellulose membranes (Pall Corporation). Blots were blocked in PBS/5% skimmed milk/0.3% Tween 20 overnight at 4° C. Afterwards they were incubated with primary antibody against Neurofilament-L (1:500) (Sigma) for 90 min at RT followed by 3 washes with PBS/0.3% Tween 20 and then 60 min with alkaline horse radish peroxidase (HRP)-labeled secondary antibody (1:20000) (Santa Cruz Biotechnology). After 3 washes with PBS/0.3% Tween 20 specific protein bands were detected by the enhanced chemiluminescence (ECL) system (Santa Cruz Biotechnology). ECL films were imaged on an Umax PowerLooklll scanner using Photoshop software (Adobe Systems), and band intensities were determined densitometrically using Quantity One software (PDI).

Immunohistochemical Staining

For immunohistochemical staining, the lumbal spinal cord segments were excised, directly frozen in liquid nitrogen and stored at −80° C. Fresh-frozen tissue sections (14 μm) were cut in a cryostat, mounted on gelatin-subbed slides, and fixed for 10 min in 4% paraformaldehyd in PBS (pH 7.4). Slides were washed with three 10-min washes in PBS and treated for 15 min with PBS containing 0.1% Triton-X 100. The sections were then blocked in 3% BSA in PBS for 1 h to reduce nonspecific binding and then incubated with pimary monoclonal antibody, anti-NFL (1:100; Sigma) and anti-m-calpain (1:50; Affinity BioReagents) for 1 h at 37° C. NF-L and m-calpain were flourescently labelled with a 1 h incubation at 37° C. with Cy3-conjugated secondary antibody (1:600; Sigma). Slides were washed with three 10-min washes in PBS after each incubation. Primary and secondary antibody incubations were performed in PBS containing 1% BSA. Slides were mounted with SlowFade Light Antifade mounting media according to manufacturer's protocol.

Determination of Calpain Activity

Calpain activity was analysed by a commercially available calpain activity assay kit (Biocat, Heidelberg, Germany), according to the manufactures instructions. The assay is based on fluorometric detection of the cleavage of the calpain substrate Ac-LLY-AFC, which emits blue light (λₘₐₓ=400 nm). Upon cleavage of the substrate by calpain, free AFC emits a yellow-green fluorescence (λₘₐₓ=505 nm), which was quantified using a fluorometer (Spectra Fluor Plus, Tecan).

### Example 1

### Chronic Inflammation Leads to Alterations in a Variety of Proteins

For the detection of inflammation responsive proteins, the zymosan induced inflammation model in rats with subsequent 2D-PAGE of the rat lumbal spinal cord has been used. Protein expression patterns of control spinal cord lysate were compared to spinal cord proteins of rats after 24, 48 or 96 h zymosan treatment, respectively. More than 500 protein spots were separated on each gel with an apparent range of molecular masses from 10 tol 00 kDa and pI values from 3 to 10, as detected by Coomassie Blue staining. Zymosan treatment led to the modification of at least 10 protein spots. The effects were most pronounced 96 h after the zymosan injection.

### Example 2

### NF-L is Time Dependently Down Regulated in the Rat Spinal Cord After Zymosan Treatment

Further investigations in this study were focussed on Spot 1 that was identified by MALDI-TOF mass spectrometry as neurofilament light chain (NF-L) with a molecular mass of 61.3 kDa and a pI at 4.63 (FIG. 1A). 2D-PAGE revealed that protein expression of NF-L was time dependently downregulated after 24 and 48 h. After 96 h. the protein spot was completely diminished as shown by Commassie staining (FIG. 1A).

In order to prove the degradation of NF-L protein expression in the spinal cord after an inflammatory stimulus we also performed Western Blot analysis using a monoclonal anti-NF-L antibody which did not cross-react with other intermediate filament proteins. As shown in FIG. 1B by Western Blot analysis, the NF-L protein was degraded due to the increasing duration of the peripheral inflammation. Densitometric analysis of NF-L protein bands in the Western Blot analysis are shown in FIG. 1C.

### Example 3

### Inflammatory Stimulation Leads to an Increase of Calpain Activity

It is well known that neurofilament degradation occurs mainly by the activity of the calcium dependent cystein-protease calpain. Therefore we examined the effects of peripheral zymosan treatment on calpain activity in the spinal cord. Calpain activity was determined by fluorometric detection of the cleavage of a synthetic calpain substrate. Peripheral inflammation increased calpain activity in the spinal cord slightly after 24 and 48 h and significantly after 96 h (p<0.05)(FIG. 2A). As enzyme inhibitor we used calpain inhibitor III (Carbobenzoxy-valinyl-phenylalaninal, MDL 28170) which is described as a potent, selective and cell-permeable inhibitor of calpain 1 and II (Chatterjee et al., 1998; Rami et al., 1997). Treatment of rats with the calpain inhibitor III (i.p.) (25 mg/kg) significantly inhibited the zymosan-induced protease activation in the spinal cord (FIG. 2B).

### Example 4

### Immunohistochemistry

Immunohistochemistry was applied to determine the protein levels of neurofilament-L and calpain in rat spinal cord slices after zymosan treatment with and without additional administration of calpain inhibitor III.

Slices treated with a specific NF-L-antibody showed reduced immunoflourescence intensity after 96 h of zymosan treatment indicating the neurofilament breakdown. This effect was reversed by addition of calpain inhibitor III. Slices treated with the protease inhibitor showed immunofluorescence levels comparable to control slices of untreated rats (FIG. 3A).

To assess calpain protein expression an antibody against m-calpain was used. m-calpain expression in spinal cord slices was slightly enhanced after zymosan treatment (96 h). This increase in immunoreactivity was not altered by application of the calpain inhibitor III (FIG. 3B)

### Example 5

### Effects of a Calpain Inhibitor in the Zymosan Induced Inflammation Model in Rats

Since calpain activity is upregulated by zymosan induced inflammation and thereby probably causes neurofilament degradation we hypothesized that the specific calpain inhibitor might provide anti-inflammatory properties. We tested this hypothesis again in the zymosan-induced paw inflammation model in rats. In vehicle treated rats intraplantar injection of 1.25 mg zymosan led to a maximum increase of the paw volume of 129±5.9 (mean±sem) % after 4 h. As hypothesized calpain inhibitor III inhibited paw inflammation dose-dependently at doses of 12.5 and 25 mg/kg (FIG. 4A). Statistical comparison of the area under the “paw volume increase” versus “time” curves (AUC_{ΔPV} from 0-8 h) revealed statistically significant differences between control rats and rats treated with 25 mg/kg calpain inhibitor (p<0.001). Results of the post hoc analysis are shown in FIG. 4B.

### Example 6

### Effects of Calpain Inhibitor III in the Zymosan Induced Thermal Hyperalgesia in Rats

To assess the influence of calpain inhibition on hyperalgesia we determined the paw withdrawal latency in the thermal hyperalgesia model. The results in FIG. 5A suggest that rats treated with calpain inhibitor III showed significantly higher paw withdrawal latency as compared to rats treated with zymosan alone indicating that the inhibitor treated rats felt less pain. Results of the post hoc analysis are shown in FIG. 5B.

Discussion of Results of Examples:

The results of the present study showed by the means of 2D gel electrophoresis that neurofilament light chain is degraded after peripheral inflammatory stimulation. Neurofilaments represent an important group of cytoskeleton proteins that are involved in the control of axonal caliber and architecture and axoplasmic flow (Perrone Capano et al., 2001). Neurofilament abnormalities provoke selective degeneration and death of motoneurons and come along with neurodegenerative diseases (Julien, 1999). Neurofilament degradation has been observed in spinal cord injury as well as under anoxic and ischemic conditions by activation of the calcium dependent cystein protease calpain (Banik et al., 1997; Leski et al., 2001; Stys et al., 2002).

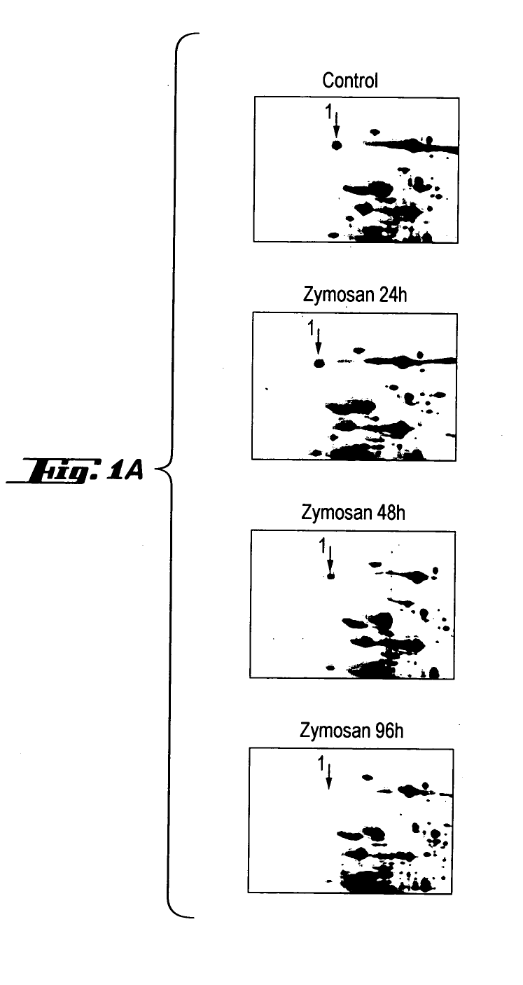

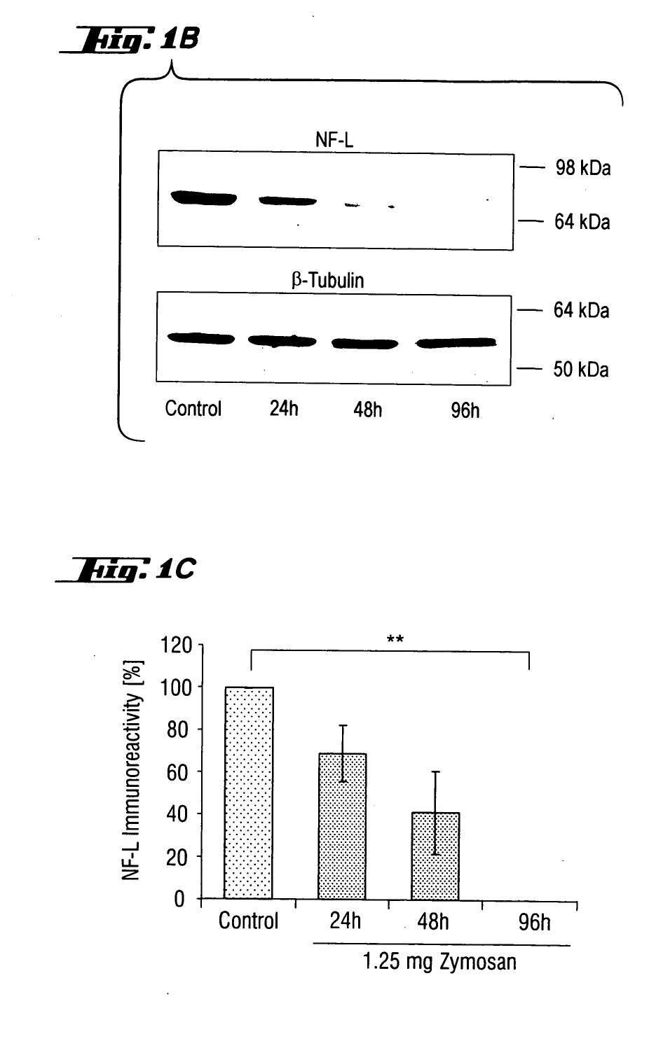

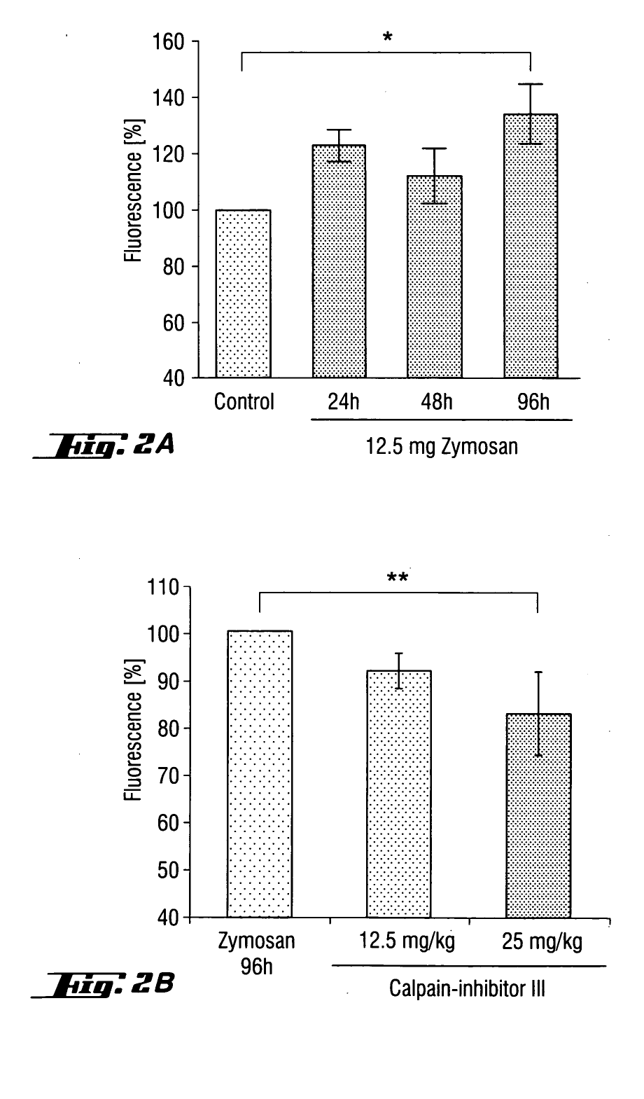

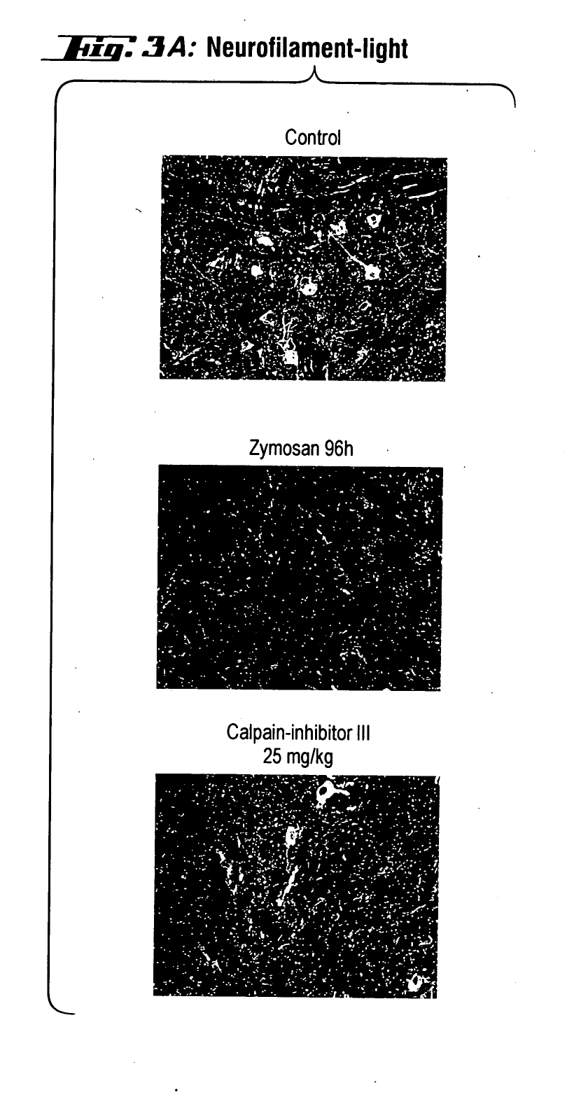

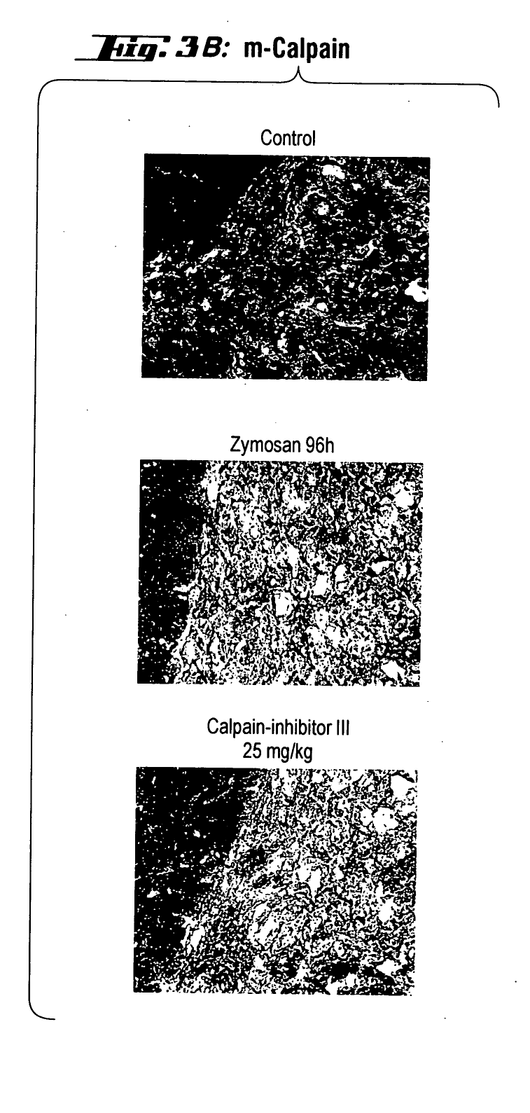

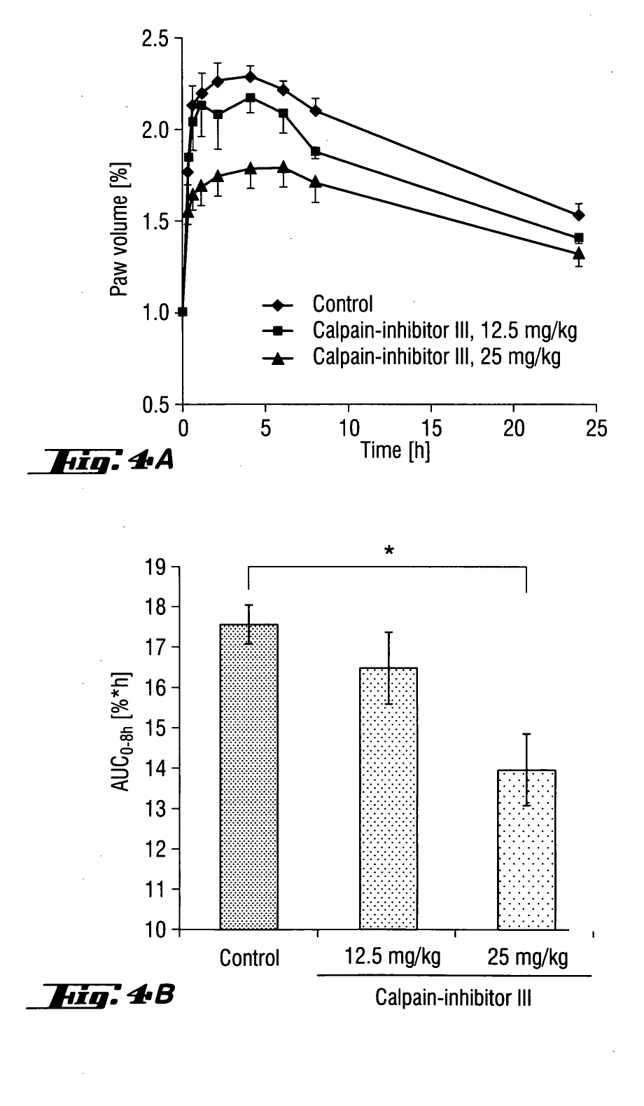

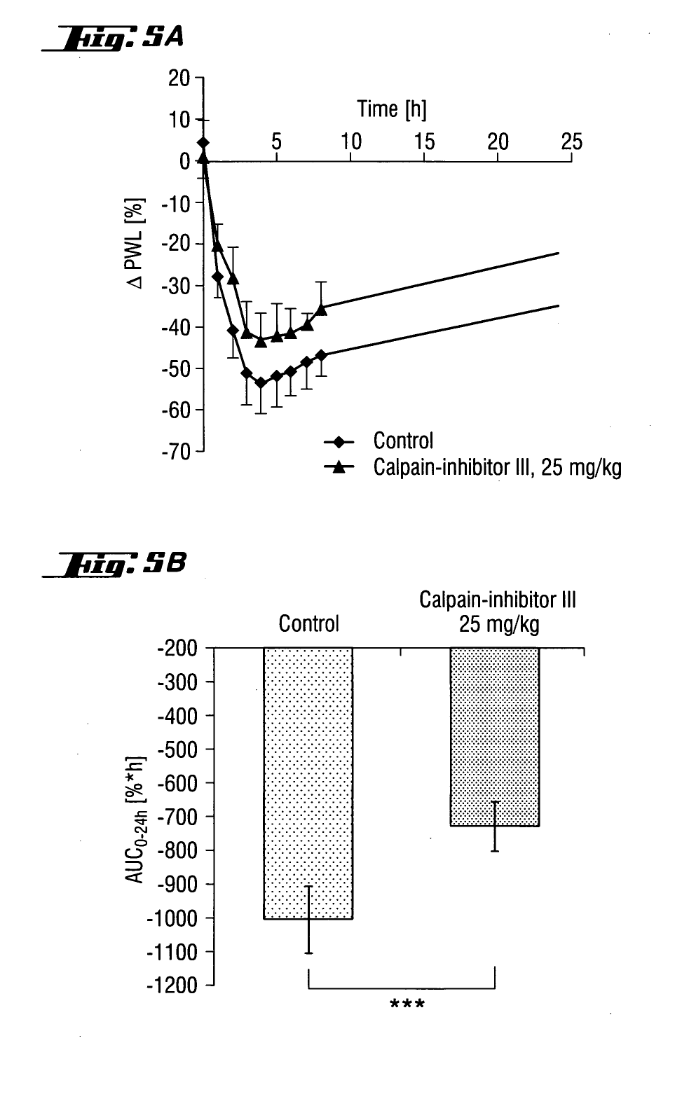

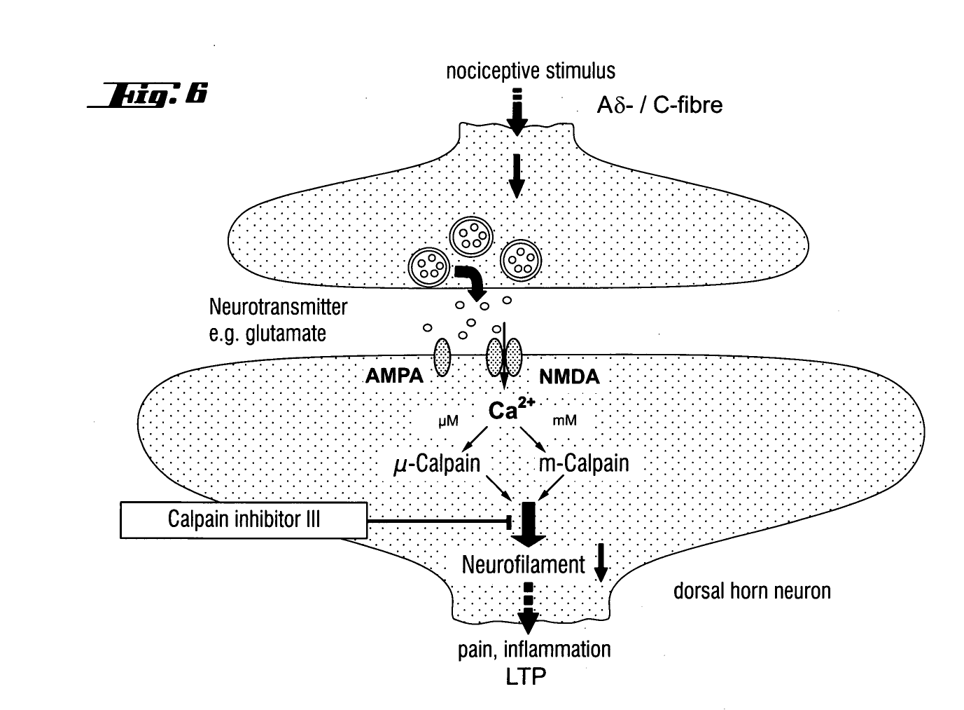

As summarized in FIG. 6, noxious peripheral stimulation, e.g. inflammatory conditions, triggers the release of neurotransmitters in the spinal cord, in particular the excitatory amino acid glutamate and the neuropeptide substance P leading to activation of voltage-gated calcium channels as well as ionotropic and metabotropic receptors, thereby provoking a calcium influx from voltage sensitive ion channels and also a calcium release from intracellular stores. The resulting calcium accumulation induces calpain activation as well as upregulation and activation of several further proteins in the spinal cord e.g. nNOS, Ca²⁺-Calmodulin Kinase (Shields et al., 1998) and adenylyl cyclase. The here found increase in calpain activity and expression may also be related to the production of cytokines and activation of the arachidonic acid cascade which occurs during inflammatory processes in astrocytes and inflammatory immune cells. In vitro studies have also shown increases of synthesis and activity of calpain in glia, neuronal and lymphoid cells in response to stress (Ray et al., 2003).

Currently, there are two major isoforms of calpain in the central nervous system, p-calpain (calpain I) and m-calpain (calpain II) which are activated by micromolar and millimolar quantities of calcium, respectively. They have been implicated in a number of physiological and pathological conditions including neuronal plasticity and neuronal cell death. With respect to this neuronal cell death, activation of calpain leads to the proteolysis of several cellular proteins, mostly associated with the cellular membrane, including cytoskeletal proteins (eg, neurofilament, spectrin, fodrin, and microtubule-associated proteins), membrane proteins (eg, growth factor receptors, adhesion molecules, and ion transporters), enzymes (eg, kinases, phosphateses, and phospholipases), as well as cytokines and transcription factors (Kampfl et al., 1997; Shields et al., 1998). Although many of these calpain actions may be implicated in mechanisms contributing to inflammation, the exact role of calpain activation in inflamed tissue remains to be clarified.

There is evidence that activation of calpain leads to the degradation of IκB in the proteasome and, hence, is an essential step in the translocation of nuclear factor κB (NF-κB) from the cytosol into the nucleus (Chen et al., 2000; Saido et al., 1994). Calpain inhibitor I has been shown to prevent this NF-kB activation and the subsequent upregulation of iNOS and COX-2 protein thereby reducing the development of acute and chronical inflammation (Cuzzocrea et al., 2000). The anti-inflammatory drug indomethacin which inhibits cyclooxygenase activity and prostaglandin upregulation showed additionally calpain inhibiting activity (Banik et al., 2000). In addition, recent findings of increased calpain activity in ischemia, Alzheimer's disease, spinal cord injury, and brain trauma have implicated calpain mediated proteolysis in tissue destruction and degeneration in CNS trauma and diseases (Banik et al., 1997; Shields et al., 1998). Uncontrolled calpain activation leads to cytoskeletal protein breakdown, subsequent loss of structural integrity and disturbances of axonal transport.

The findings of increased calpain activity in diseases suggest that calpain inhibitors may be employed as therapeutic agents since they minimize tissue degeneration by preventing degradation of substrate proteins. In our study we used Calpain inhibitor Ill (MDL 28170), a potent, specific calpain inhibitor that rapidly penetrates the blood-brain barrier following systemic administration. Intraperitoneal application of this inhibitor blocked the degradation of neurofilament protein in the spinal cord which was due to inhibition of the protease activity but not to calpain protein downregulation. Additional effects of calpain inhibition on other calpain substrates are also possible but have not been investigated in this study.

For the first time we could show that pre-treatment of rats with calpain inhibitor III attenuates the development of zymosan induced paw inflammation and thermal hyperalgesia in rats. In conclusion, we propose that calpain is an important mediator in nociceptive responses and that calpain inhibitor III may be useful in the therapy of inflammatory diseases.

### Literature

Banik, N. L., Matzelle, D., Terry, E., Gantt-Wilford, G. & Hogan, E. L.
   Inhibition of proteolysis by a cyclooxygenase inhibitor, indomethacin. Neurochem Res, 25, 1509-15(2000)
Banik, N. L., Matzelle, D. C., Gantt-Wilford, G., Osborne, A. & Hogan, E. L.
   Increased calpain content and progressive degradation of neurofilament protein in spinal cord injury. Brain Res, 752, 301-6(1997)
Beiche, F., Scheuerer, S., Brune, K., Geisslinger, G. & Goppelt-Struebe, M.
   Up-regulation of cyclooxygenase-2 mRNA in the rat spinal cord following peripheral inflammation. FEBS Lett, 390, 165-9(1996)
Bliss, T. V. & Collingridge, G. L.
   A synaptic model of memory: long-term potentiation in the hippocampus. Nature, 361, 31-9(1993)
Chan, S. L. & Mattson, M. P.
   Caspase and calpain substrates: roles in synaptic plasticity and cell death. J Neurosci Res, 58, 167-90 Order (1999)
Chatterjee, S., Gu, Z. Q., Dunn, D., Tao, M., Josef, K., Tripathy, R., Bihovsky, R., Senadhi, S. E., O'Kane, T. M., McKenna, B. A., Mallya, S., Ator, M. A., Bozyczko-Coyne, D., Siman, R. & Mallamo, J. P.
   D-amino acid containing, high-affinity inhibitors of recombinant human calpain I. J Med Chem, 41, 2663-6(1998)
Chen, F., Demers, L. M., Vallyathan, V., Lu, Y., Castranova, V. & Shi, X.
   Impairment of NF-kappaB activation and modulation of gene expression by calpastatin. Am J Physiol Cell Physiol, 279, C709-16(2000)
Cuzzocrea, S., McDonald, M. C., Mazzon, E., Siriwardena, D., Serraino, I., Dugo, L., Britti, D., Mazzullo, G., Caputi, A. P. & Thiemermann, C.
   Calpain inhibitor I reduces the development of acute and chronic inflammation. Am J Pathol, 157, 2065-79(2000)
Dubner, R. & Ruda, M. A.
   Activity-dependent neuronal plasticity following tissue injury and inflammation. Trends Neurosci, 15, 96-103(1992)
Gorg, A., Boguth, G., Obermaier, C., Posch, A. & Weiss, W.
   Two-dimensional polyacrylamide gel electrophoresis with immobilized pH gradients in the first dimension (IPG-Dalt): the state of the art and the controversy of vertical versus horizontal systems. Electrophoresis, 16, 1079-86(1995)
Hargreaves, K., Dubner, R., Brown, F., Flores, C. & Joris, J.
   A new and sensitive method for measuring thermal nociception in cutaneous hyperalgesia. Pain, 32, 77-88(1988)
Ji, R. R., Zhang, X., Wiesenfeld-Hallin, Z. & Hokfelt, T.
   Expression of neuropeptide Y and neuropeptide Y (Y1) receptor mRNA in rat spinal cord and dorsal root ganglia following peripheral tissue inflammation. J Neurosci, 14, 6423-34(1994)
Ji, R. R., Zhang, X., Zhang, Q., Dagerlind, A., Nilsson, S., Wiesenfeld-Hallin, Z. & Hokfelt, T.
   Central and peripheral expression of galanin in response to inflammation. Neuroscience, 68, 563-76(1995)
Julien, J. P.
   Neurofilament functions in health and disease. Curr Opin Neurobiol, 9, 554-60(1999)
Kampfl, A., Posmantur, R. M., Zhao, X., Schmutzhard, E., Clifton, G. L. & Hayes, R. L.
   Mechanisms of calpain proteolysis following traumatic brain injury: implications for pathology and therapy: implications for pathology and therapy: a review and update. J Neurotrauma, 14, 121-34(1997)
Leski, M. L., Bao, F., Wu, L., Qian, H., Sun, D. & Liu, D.
   Protein and DNA oxidation in spinal injury: neurofilaments—an oxidation target. Free Radic Biol Med, 30, 613-24(2001)
Mannion, R. J., Costigan, M., Decosterd, I., Amaya, F., Ma, Q. P., Holstege, J. C., Ji, R. R., Acheson, A., Lindsay, R. M., Wilkinson, G. A. & Woolf, C. J.
   Neurotrophins: peripherally and centrally acting modulators of tactile stimulus-induced inflammatory pain hypersensitivity. Proc Natl Acad Sci USA, 96, 9385-90(1999)
McCarson, K. E. & Krause, J. E.
   NK-1 and NK-3 type tachykinin receptor mRNA expression in the rat spinal cord dorsal horn is increased during adjuvant or formalin-induced nociception. J Neurosci, 14, 712-20(1994)
Perrone Capano, C., Pernas-Alonso, R. & di Porzio, U.
   Neurofilament homeostasis and motoneurone degeneration. Bioessays, 23, 24-33(2001)
Rami, A., Ferger, D. & Krieglstein, J.
   Blockade of calpain proteolytic activity rescues neurons from glutamate excitotoxicity. Neurosci Res, 27, 93-7(1997)
Ray, S. K., Hogan, E. L. & Banik, N. L.
   Calpain in the pathophysiology of spinal cord injury: neuroprotection with calpain inhibitors. Brain Res Brain Res Rev, 42, 169-85 Order (2003)
Ray, S. K., Matzelle, D. C., Wilford, G. G., Hogan, E. L. & Banik, N. L.
   E-64-d prevents both calpain upregulation and apoptosis in the lesion and penumbra following spinal cord injury in rats. Brain Res, 867, 80-9 Order (2000a)
Ray, S. K., Wilford, G. G., Ali, S. F. & Banik, N. L.
   Calpain upregulation in spinal cords of mice with 1-methyl-4-phenyl-1,2,3,6-tetrahydropyridine (MPTP)-induced Parkinson's disease. Ann N Y Acad Sci, 914, 275-83(2000b)
Rygh, L. J., Svendsen, F., Hole, K. & Tjolsen, A.
   Natural noxious stimulation can induce long-term increase of spinal nociceptive responses. Pain, 82, 305-10(1999)
Saido, T. C., Sorimachi, H. & Suzuki, K.
   Calpain: new perspectives in molecular diversity and physiological-pathological involvement. Faseb J, 8, 814-22(1994)
Sakaguchi, T., Okada, M., Kitamura, T. & Kawasaki, K.
   Reduced diameter and conduction velocity of myelinated fibers in the sciatic nerve of a neurofilament-deficient mutant quail. Neurosci Lett, 153, 65-8 Order (1993)
Schlaepfer, W. W., Lee, C., Lee, V. M. & Zimmerman, U. J.
   An immunoblot study of neurofilament degradation in situ and during calcium-activated proteolysis. J Neurochem, 44, 502-9(1985)
Shields, D. C., Tyor, W. R., Deibler, G. E., Hogan, E. L. & Banik, N. L.
   Increased calpain expression in activated glial and inflammatory cells in experimental allergic encephalomyelitis. Proc Natl Acad Sci USA, 95, 5768-72(1998)
Stys, P. K. & Jiang, Q.
   Calpain-dependent neurofilament breakdown in anoxic and ischemic rat central axons. Neurosci Lett, 328, 150-4(2002)
Vikman, K. S., Kristensson, K. & Hill, R. H.
   Sensitization of dorsal horn neurons in a two-compartment cell culture model: wind-up and long-term potentiation-like responses. J Neurosci, 21, RC169(2001)
Woolf, C. J. & Costigan, M.
   Transcriptional and posttranslational plasticity and the generation of inflammatory pain. Proc Natl Acad Sci USA, 96, 7723-30(1999)
Yaksh, T. L., Hua, X. Y., Kalcheva, I., Nozaki-Taguchi, N. & Marsala, M.
   The spinal biology in humans and animals of pain states generated by persistent small afferent input. Proc Natl Acad Sci USA, 96, 7680-6(1999)

1. A method of screening pharmaceuticals useful for modulating and/or preventing pain, comprising the steps
a. providing two samples
b. contacting one sample containing calpain or a functional fragment or derivative thereof with a compound,
c. determining the calpain activity in the presence of compound,
d. determining the calpain activity in the absence of compound, and
e. comparing the calpain activity according to c) with that according to d).

2. The method according to claim 1, wherein calpain is selected from the group consisting of human calpain I and calpain II.

3. The method according to claim 1, wherein calpain is an isolated polypeptide or polynucleotide.

4. The method according to claim 3, wherein calpain is an isolated polypeptide.

5. The method according to claim 4, wherein calpain is a polypeptide comprising an amino acid sequence selected from the group consisting of SEQ ID No 1, a functional fragment of SEQ ID No 1, SEQ ID No 3, and a functional fragment of SEQ ID No3.

6. The method according to claim 3, wherein calpain is an isolated polynucleotide.

7. The method according to claim 6, wherein calpain is a polynucleotide comprising a sequence selected from the group consisting of SEQ ID No 2, SEQ ID No 4, functional fragments of SEQ ID No 2, functional fragments of SEQ ID No 4, a polynucleotide capable of hybridizing to SEQ ID No 2 under stringent conditions and a polynucleotide capable of hybridizing to SEQ ID No 4 under stringent conditions.

8. The method according to claim 5, wherein calpain is a polypeptide comprising amino acids 115 to 272 of SEQ ID No 1

9. The method according to claim 5, wherein calpain is a polypeptide comprising amino acids 105 to 262 of SEQ ID No 3.

10. The method according to claim 1, wherein the sample containing calpain is a cell expressing calpain.

11. The method according to claim 10, wherein the sample containing calpain is a cell expressing recombinant calpain.

12. The method according to claim 10, wherein the cell expressing calpain has been modified to have a lower calpain activity as compared to its unmodified state.

13. The method according to claim 14, wherein a calpain knock-out cell expressing recombinant calpain is used.

14. The method according to claim 1, wherein the calpain activity is determined directly.

15. The method according to claim 1, wherein the calpain activity is determined indirectly.

16. A method of identifying a compound that modulates pain comprising
a. Selecting a compound that modulates the activity of calpain as a test compound, and
b. Administering said test compound to a subject to determine whether the pain is modulated.

17. A method of treating pain comprising admistering a pain-ameliorating amount of MDL-28170 to a patient in need of pain treatment.

18. A method of treating pain comprising administering a pain-ameliorating amount of MDL-102935 to a patient in need of pain treatment.
